SEQUENCE LISTING (11 sequences)

SEQ ID NO: 1 - DNA, 1676 bp, Homo sapiens
agccttatggattggattcgactgaccaaaagtggaaaggatctaacgggattaaaaggcaggttaattgaagtaactgaagaagaacttaagaaacacaacaaaaaagatgattgttggatatgcataagaggtttcgtttataatgtcagcccttatatggagtatcatcctggtggagaagatgaactaatgagagcagcaggatcagatggtactgaactttttgatcaggttcatcgttgggtcaattatgaatccatgctgaaagaatgcctggttggcagaatggccattaaacctgctgttctgaaagactatcgtgaggaggaaaagaaagtcttaaatggcatgcttcccaagagccaagtgacagatacacttgccaaagaaggtcctagttatccaagctatgattggttccaaacagactctttagtcaccattgccatatatactaaacagaaggatatcaatttagactcaattatagttgatcatcagaatgattcctttagagcagaaacaattattaaggattgtttatatcttatacatattgggctaagccatgaggttcaggaagatttttctgtgcgggttgttgagagtgtgggaaaaatagagattgttctacaaaaaaaagagaatacttcttgggactttcttggccatcccctgaagaatcataattcacttattccaaggaaagatacaggtttgtactacagaaagtgccagttaatttccaaggaagatgttactcatgatacgaggcttttctgtttgatgctgccaccaagcactcatcttcaagtgcccattgggcaacatgtttacctcaagctacctattacaggtacagaaatagtaaagccatatacacctgtatctggttccttactctcagagttcaaggaaccagttcttcccaacaataaatacatctactttttgataaaaatctatcccactggactcttcacaccagagcttgatcgtcttcagattggagattttgtttctgtaagcagtcctgagggcaattttaaaatatccaagttccaagaattagaagatctctttttgttggcagctggaacaggcttcacaccaatggttaaaatactgaattatgctttgactgatatacccagtctcaggaaagtgaagctgatgttcttcaataaaacagaagatgatataatttggagaagccaattggagaaattagcatttaaagataaaagactggatgttgaatttgttctctcagcacctatttctgaatggaatggcaaacagggacatatttcaccagctcttctttctgaatttttgaaaagaaatttggacaaatccaaagttctcgtctgcatttgtggaccagtgccatttacagaacaaggagtaaggttgctgcatgatctcaacttttccaaaaatgagatccatagttttacagcataatgaagagctgtcattgtcctttattcaactagtttatctaaatttgtgattgcttagggttttttaagagaacatttttgtacataacaaaaggttaactagaatccagccttcagtttcttaaatgaaatcaaatgttccttcagtacaggtaacttcttggctttcttttgtaccacaacttattttactactgatatttgacc

SEQ ID NO: 2 - protein, 487 aa, Homo sapiens
MDWIRLTKSGKDLTGLKGRLIEVTEEELKKHNKKDDCWICIRGFVYNVSPYMEYHPGGEDELMRAAGSDGTELFDQVHRWVNYESMLKECLVGRMAIKPAVLKDYREEEKKVLNGMLPKSQVTDTLAKEGPSYPSYDWFQTDSLVTIAIYTKQKDINLDSIIVDHQNDSFRAETIIKDCLYLIHIGLSHEVQEDFSVRVVESVGKIEIVLQKKENTSWDFLGHPLKNHNSLIPRKDTGLYYRKCQLISKEDVTHDTRLFCLMLPPSTHLQVPIGQHVYLKLPITGTEIVKPYTPVSGSLLSEFKEPVLPNNKYIYFLIKIYPTGLFTPELDRLQIGDFVSVSSPEGNFKISKFQELEDLFLLAAGTGFTPMVKILNYALTDIPSLRKVKLMFFNKTEDDIIWRSQLEKLAFKDKRLDVEFVLSAPISEWNGKQGHISPALLSEFLKRNLDKSKVLVCICGPVPFTEQGVRLLHDLNFSKNEIHSFTA

SEQ ID NO: 3 - protein, 135 aa, Homo sapiens
MDWIRLTKSGKDLTGLKGRLIEVTEEELKKHNKKDDCWICIRGFVYNVSPYMEYHPGGEDELMRAAGSDGTELFDQVHRWVNYESMLKECLVGRMAIKPAVLKDYREEEKKVLNGMLPKSQVTDTLAKEGPSYPS

SEQ ID NO: 4 - protein, 90 aa, Homo sapiens
YDWFQTDSLVTIAIYTKQKDINLDSIIVDHQNDSFRAETIIKDCLYLIHIGLSHEVQEDFSVRVVESVGKIEIVLQKKENTSWDFLGHPL

SEQ ID NO: 5 - protein, 262 aa, Homo sapiens
KNHNSLIPRKDTGLYYRKCQLISKEDVTHDTRLFCLMLPPSTHLQVPIGQHVYLKLPITGTEIVKPYTPVSGSLLSEFKEPVLPNNKYIYFLIKIYPTGLFTPELDRLQIGDFVSVSSPEGNFKISKFQELEDLFLLAAGTGFTPMVKILNYALTDIPSLRKVKLMFFNKTEDDIIWRSQLEKLAFKDKRLDVEFVLSAPISEWNGKQGHISPALLSEFLKRNLDKSKVLVCICGPVPFTEQGVRLLHDLNFSKNEIHSFTA

SEQ ID NO: 6 - protein, 494 aa, Mus musculus
MDWIRLTKSGKDLTGLKGGLIEVTEEELKKHNKKEDCWICIRGFVYNVSPYMEYHPGGEDELMRAAGADGTDLFNEVHRWVNYESMLKECLVGRMAVKPAVPKDCHEGKRVLNGMLPKSQMSDTLPRDVTDTLPREGLSSPSYDWFQTESSVTIVVYTKQKNISLDSVIVDLQDDSLRAEAVIKDHSYLVHVGLSHEVQENFSVRVIENVGKIEIVLQKKESVSWQCLGDHLEKHDSFIPKKDTGLYYRRCQLISKEDVTHDTRLLCLMLPPSTHLQVPVGQHVYLKLSVTGAEIVKPYTPVSDSLLSDFKEPVLSPNKYIYFLIKIYPAGLFTPELDRLQIGDFISVSGPEGNFKVSKLQEVEDLFLLAAGTGFTPMVTVLNYALSHMSSLRKVKLMFFNKTEDDIIWRCQLEKLALREKRFDVEFVLSAPSPEWNGKQGHISRALLSEFLQRSSENSRAFLCICGPTPFTDEGIRLLHDLNFSDDEIHGFTA

SEQ ID NO: 7 - protein, 486 aa, Rattus norvegicus
MDWFRLTKSGKDFTGLKGGLIEVTEEELKKHNKKDDCWICIRGFVYNVSPYMEYHPGGEDELMRAAGADGTDLFNEVHRWVNYESMLKECLVGRMAVKPAVPKDCHEGKRVLNGMLPKSQVTDTLPREGPSSPSYDWFQTESSVTIVIYTKQKNINLDSVIVDLQDDSLRAEAVIKDHSYLIHIGLSHEVQENFSVRVIENVGKIEIVLLKKETVSWKCLGDPLEKHDSFIPKKDTGLYYRQCQLISKEDVTHDTRLFCLMLPPSTHLQVPVGQHVYLKLSVTGAEIVKPYTPVSESLLSDFKEPVLSPNKYIYFLIKIYPAGLFTPELDRLQIGDFVSVSGPEGNFKVSKLQEVEDLFLLAAGTGFTPMVTVLNHALTHMSSLRKVKLMFFNKTEDDIIWRCQLEKLALKDKRFHVEYVLSAPSPEWNGKQGHVSRALLSEFLQRSLENSKVFLCICGPTPFTDEGIRLLHDLNFSDDEIHGFTA

SEQ ID NO: 8 - protein, 495 aa, Artificial Sequence
  Description of Artificial Sequence consensus sequence
MDWXRLTKSGKDXTGLKGXLIEVTEEELKKHNKKXDCWICIRGFVYNVSPYMEYHPGGEDELMRAAGXDGTXLFXXVHRWVNYESMLKECLVGRMAXKPAVXKDXXEXXKXVLNGMLPKSQXXXXXXXXVTDTLXXEGXSXPSYDWFQTXSXVTIXXYTKQKXIXLDSXIVDXQXDSXRAEXXIKDXXYLXHXGLSHEVQEXFSVRVXEXVGKIEIVLXKKEXXSWXXLGXXLXXHXSXIPXKDTGLYYRXCQLISKEDVTHDTRLXCLMLPPSTHLQVPXGQHVYLKLXXTGXEIVKPYTPVSXSLLSXFKEPVLXXNKYIYFLIKIYPXGLFTPELDRLQIGDFXSVSXPEGNFKXSKXQEXEDLFLLAAGTGFTPMVXXLNXALXXXXSLRKVKLMFFNKTEDDIIWRXQLEKLAXXXKRXXVEXVLSAPXXEWNGKQGHXSXALLSEFLXRXXXXSXXXXCICGPXPFTXXGXRLLHDLNFSXXEIHXFTA

SEQ ID NO: 9 - protein, 135 aa, Artificial Sequence
  Description of Artificial Sequence consensus sequence
MDWXRLTKSGKDXTGLKGXLIEVTEEELKKHNKKXDCWICIRGFVYNVSPYMEYHPGGEDELMRAAGXDGTXLFXXVHRWVNYESMLKECLVGRMAXKPAVXKDXXEXXKXVLNGMLPKSQXXXXXXXXVTDTLX

SEQ ID NO: 10 - protein, 105 aa, Artificial Sequence
  Description of Artificial Sequence consensus sequence
XEGXSXPSYDWFQTXSXVTIXXYTKQKXIXLDSXIVDXQXDSXRAEXXIKDXXYLXHXGLSHEVQEXFSVRVXEXVGKIEIVLXKKEXXSWXXLGXXLXXHXSXI

SEQ ID NO: 11 - protein, 255 aa, Artificial Sequence
  Description of Artificial Sequence consensus sequence
PXKDTGLYYRXCQLISKEDVTHDTRLXCLMLPPSTHLQVPXGQHVYLKLXXTGXEIVKPYTPVSXSLLSXFKEPVLXXNKYIYFLIKIYPXGLFTPELDRLQIGDFXSVSXPEGNFKXSKXQEXEDLFLLAAGTGFTPMVXXLNXALXXXXSLRKVKLMFFNKTEDDIIWRXQLEKLAXXXKRXXVEXVLSAPXXEWNGKQGHXSXALLSEFLXRXXXXSXXXXCICGPXPFTXXGXRLLHDLNFSXXEIHXFTA

(19) United States
(12) Patent Application Publication
(10) Pub. No.: US 2005/0031605 A1
(43) Pub. Date: Feb. 10, 2005

(54) Compositions and methods of treating diabetes

(76) Inventors: Howard F. Bunn, Auburndale, MA (US); Jianxin Xie, Waltham, MA (US); Hao Zhu, Bedford, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/772,076

(22) Filed: Feb. 3, 2004

Related U.S. Application Data
(60) Provisional application No. 60/444,784, filed on Feb. 3, 2003.

(51) Int. Cl. A61K 48/00; A61K 38/44

(52) U.S. Cl. 424/94.4; 424/93.21; 514/44

(57) ABSTRACT

The invention features compositions and methods for increasing insulin production or decreasing blood glucose levels. Also included are methods of treating diabetes and obesity.

### RELATED APPLICATIONS

This application claims priority to U.S. Ser. No. 60/444,784 filed Feb. 3, 2003 which is incorporated herein by reference in its entirety.

### TECHNICAL FIELD

This invention relates to diabetes.

### BACKGROUND OF THE INVENTION

Diabetes is generally classified in two main groups. In type 1 diabetes, auto-immune destruction of β-cells within the islets of Langerhans leads to a marked defect in insulin production. In contrast, type 2 diabetes is characterized by insulin resistance in muscle, fat, and liver along with a relative impairment of insulin production in β-cells. Multiple genes contribute to susceptibility in both type 1 and type 2 diabetes, although in most cases their identities remain unknown.

### SUMMARY

The invention is based on the discovery that a decrease in the oxidoreductase, Ncb5or, leads to insulin deficiency in mice. Accordingly, the invention features methods of increasing insulin production by contacting a cell with compound that increases the expression or activity of a flavo-heme oxido-reductase polypeptide. An increase of oxidoreductase expression or activity is defined by superoxide production. For example, activity of an oxidoreductase polypeptide is measured by detecting superoxide production in the presence of air and excess NAD(P)H or by cytochrome C reduction. The cell is any cell that is capable of expressing insulin, e.g., the cell is a pancreatic cell such as a pancreatic islet cell. The pancreatic islet cell is a beta cell, or alternatively, an alpha cell. The cell is contacted in vivo, in vitro, or ex vivo.

Insulin is produced in the “prohormone” form. Alternatively, the insulin is in the fully processed biologically active form of the hormone. By biologically active form is meant a fully processed form of insulin capable of promoting, e.g., glucose utilization, carbohydrate, fat and protein metabolism. Methods of measuring insulin production are well known in the art and include, e.g., immunoassays using insulin-specific antibodies.

The invention also features methods of alleviating a symptom of diabetes, e.g., increasing serum insulin levels or decreasing serum glucose levels in a subject, by administering to the subject a compound that increases a flavo-heme oxido-reductase polypeptide expression or activity. The subject is a mammal such as human, a primate, mouse, rat, dog, cat, cow, horse, pig.

The subject is suffering from or at risk of developing diabetes. A subject suffering from or at risk of developing diabetes is identified by methods known in the art such as determining blood glucose levels. For example, a blood glucose value above 140 mg/dL on at least two occasions after an overnight fast means a person has diabetes. A person not suffering from or at risk of developing diabetes is characterized as having fasting sugar levels between 70-110 mg/dL.

Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia, insulin deficiency or resistance. Diabetic or pre-diabetic patients to which the compounds are administered are identified using diagnostic methods know in the art.

The invention further features a method of inhibiting the loss of a beta cell in pancreatic islet tissue by contacting pancreatic islet tissue with a compound that increases the expression or activity of a flavo-heme oxido-reductase polypeptide. By decreasing the loss is meant that the pancreatic tissue has 10%, 20%, 30%, 40% or more beta cells in the presence of the compound compared to the absence of the compound. The amount of reactive oxygen species is the pancreatic tissue is reduced in the presence of the compound as compared to the absence of the compound. The reactive oxygen species is superoxide or ferri-heme. Optionally, the pancreatic islet tissue is further contacted with a anti-oxidant. The anti-oxidant is a niacin compound such as nicotimamide.

The invention also features a method of increasing the viability or proliferation of pancreatic islet cells by contacting a cell with a compound that increases the expression or activity of a flavo-heme oxido-reductase polypeptide. Additionally, viability of pancreatic islet cells is increased by administering to a transplant recipient a compound that increases the expression or activity of a flavo-heme oxido-reductase polypeptide. The pancreatic islet cells are primary islet cells. Alternatively, the cells are transplanted donor pancreatic cells. By viability is meant that the cell is excludes a vital dye, such as trypan. Viable cells are also capable of proliferation, differentiation, growth and development. Viability is measured by methods known in the art such as trypan blue staining. The cells are contacted in vivo, in vitro or ex vivo. The compound is administered locally to a transplanted site. Alternatively the compound is administered systemically. The compound is administered to the transplant recipient prior to or after transplantation of donor pancreatic islet cells. Optionally, the compound is administered to the transplant recipient concurrently with the transplantation of donor pancreatic

Also included in the invention are methods of inhibiting cell death by contacting the cell with a with compound that increases the expression or activity of a flavo-heme oxido-reductase polypeptide. The cells are contacted in vivo, in vitro or ex vivo. The cell is a pancreatic cell such as a pancreatic islet β-cell. The cell death is oxidative stress induced cell death or apoptotic cell death

The compound is a flavo-heme oxido-reductase polypeptide or a nucleic acid encoding a flavo-heme oxido-reductase polypeptide. Alternatively, the compound is an agonist of a flavo-heme oxido-reductase polypeptide or an inducer of the expression of a flavo-heme oxido-reductase nucleic acid. An agonist mimics at least one activity of the naturally occurring Ncb5or enzyme. An agonist compound is preferably a small molecule. An inducer is a compound that upregulates Ncb5or expression, e.g., by increasing the level of transcription or by increasing the stability of transcripts. The polypeptide or polypeptide agonist binds to a fatty acid. The fatty acid is unsaturated or saturated. Binding to a fatty acid is measured by methods known in the art.

Preferably, the flavo-heme oxido-reductase polypeptide or nucleic acid is Ncb5or. Exemplary oxido-reductase nucleic acids include the gene encoding human Ncb5or (and the encoded protein sequence; GENBANK™ Accession Nos.: AF169803 (SEQ ID No:1) and AAF04812 (SEQ ID NO:2)). The nucleic acid is operatively linked to a promoter. The promoter directs expression of the nucleic acid in the cell. For example, the promoter is a pancreatic cell-specific promoter such as an insulin promoter. Alternatively, a portion of the Ncb5or nucleic acid or polypeptide is used, such as the regions corresponding to the cyt b5, hinge region, or cyt brR domain. For example, the polypeptide contains amino acids 1-135 (SEQ ID NO:3), 136-225 (SEQ ID NO:4), and/or 226-487 (SEQ ID NO:5) of a Ncb5or polypeptide.

Also included in the invention are methods of diagnosing diabetes or a predisposition to diabetes in a subject by detecting a mutation in a gene encoding Ncb5or. The presence a mutation indicates a diagnosis of diabetes or a predisposition to diabetes. The mutation is a deletion, insertion or substitution of one or more nucleotides. The mutation is in the hinge region, i.e., amino acids 136-225 of SEQ ID NO: 2 (SEQ ID NO:4). For example, the mutation decreases the production of the polypeptide or an enzymatic activity of the polypeptide. Alternatively, the mutation is in a Ncb5or regulatory region (e.g., a mutation that leads to a decrease in Ncb5or protein production compared to a wild-type control). Diabetes or a predisposition thereto is determined by measuring the level of Ncb5or nucleic acid, polypeptide or enzyme activity in a patient-derived bodily tissue, such as blood. A decrease in the level compared to a normal control level indicates a diagnosis of diabetes or a predisposition thereto.

The invention also features a method of reducing white fat in a subject, by administering to the subject a compound, which decreases the expression or activity of Ncb5or. For example, the compound is an antisense Ncb5or nucleic acid, a Ncb5or-specific short-interfering RNA, or a Ncb5or-specific ribozyme. The compound is an inhibitor of oxidoreductase activity such as an iodonium compound, e.g., diphenyl iodonium. Preferably, the compound preferentially inhibits an enzymatic activity of Ncb5or compared to other oxidoreductase enzymes. White fat is preferentially reduced compared to brown fat.

Also included in the invention is a pharmaceutical composition that includes a Ncb5or polypeptide or a Ncb5or nucleic acid. In some aspects the composition a further includes a fatty acid.

The invention also includes polypeptides that have 80%, 85%, 90%, 95%, 98% identity to the polypeptide of SEQ ID NO:2. Identity is measures by Clustal W. Alternatively identity is measured by methods known in the art such as FASTA or BLAST analysis.

In another aspect the invention provides a method of identifying an agent that increases insulin production. The method includes contacting a cell containing a Ncb5or polypeptide or nucleic acid with a test agent and determining the level of oxidase activity in the cell. An increase in oxidase activity in the presence of the agent compared to the level in the absence of the agent indicates that the agent increases insulin production. A method of identifying a compound that increases Ncb5or gene transcription is carried out by contacting a cell containing an Ncb5or gene sequence, e.g., an Ncb5or promoter sequence, with a candidate compound. Gene transcription in the presence and in the absence of the compound is measured. An increase in gene transcription in the presence of the compound compared to that in the absence indicates the compound increases Ncb5or gene expression, i.e., the compound is an inducer of Ncb5or transcription.

In a further aspect the invention provides a method of identifying an agent that decreases fat accumulation or fat cell differentiation. The method includes contacting a cell containing a Ncb5or polypeptide or nucleic acid with a test agent and determining the level of oxidase activity in the cell. An increase in oxidase activity in the presence of the agent compared to the level in the absence of the agent indicates that the agent decreases fat accumulation. A method of identifying a compound that decreases Ncb5or gene transcription is carried out by contacting a cell containing an Ncb5or gene sequence, e.g., an Ncb5or promoter sequence, with a candidate compound. Gene transcription in the presence and in the absence of the compound is measured. An decrease in gene transcription in the presence of the compound compared to that in the absence indicates the compound is an inhibitor of Ncb5or gene expression,

The invention also includes a transgenic mouse having a homozygous disruption in a Ncb5or gene.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a photograph of a blot showing RT PCR analysis of expression of Ncb5or in whole rat embryo 14 days post conception and in organs of embryo 18 days post conception.

FIG. 2A is a diagram showing the Ncb5or wild-type allele, the knockout targeting construct and the targeted allele.

FIG. 2B is a photograph of a blot showing genotyping of mice by multiplex PCR.

FIG. 2C is a photograph of a Western blot showing Ncb5or expression in pancreata.

FIG. 2D is a photograph depicting expression of Ncb5or mRNA in isolated islets of +/+ mice.

FIG. 2E is a photograph of a Northern blot and RT-PCR analyses of Ncb5or mRNA in liver and kidney. The mRNA detected in −/− mice was derived from the knockout allele which lacks the entire exon4. WT=wild type. HT=heterozygote. KO=knockout.

FIG. 3 is a bar graph showing blood glucose levels of Ncb5or −/− mice in the fed state and the fasting state.

FIG. 4A is a bar chart showing blood glucose levels of 4 week old male Ncb5or+/+, +/−, and −/− mice. N=7-9 mice in each group. Error bars designate mean+/−SEM. *=p<0.05, **=p<0.01 and ***=p<0.001, unpaired two-tailed t test.

FIG. 4B is a bar chart showing serum insulin levels of 4 week old male Ncb5or+/+, +/−, and −/− mice. N=7-9 mice in each group. Error bars designate mean+/−SEM. *=p<0.05, **=p<0.01 and ***=p<0.001, unpaired two-tailed t test.

FIG. 4C is a line graph showing glucose tolerance test on 4 week old male Ncb5or+/+, +/, and −/− mice. N=7-9 mice in each group. Error bars designate mean+/−SEM. *=p<0.05, **=p<0.01 and ***=p<0.001, unpaired two-tailed t test.

FIG. 4D is a bar chart showing blood glucose levels of 7 week old male Ncb5or+/+, +/−, and −/− mice. N=7-9 mice in each group. Error bars designate mean+/−SEM. *=p<0.05, **=p<0.01 and ***=p<0.001, unpaired two-tailed t test.

FIG. 4E is a bar chart showing serum insulin levels of 7 week old male Ncb5or+/+, +/−, and −/− mice. N=7-9 mice in each group. Error bars designate mean+/−SEM. *=p<0.05, **=p<0.01 and ***=p<0.001, unpaired two-tailed t test.

FIG. 4F is a line graph showing glucose tolerance test on 7 week old male Ncb5or+/+, +/, and −/− mice. N=7-9 mice in each group. Error bars designate mean+/−SEM. *=p<0.05, **=p<0.01 and ***=p<0.001, unpaired two-tailed t test.

FIG. 4G is a line graph showing food intake of 7-9 week old male Ncb5or+/+, +/, and −/− mice. N=7-9 mice in each group. Error bars designate mean+/−SEM. *=p<0.05, **=p<0.01 and ***=p<0.001, unpaired two-tailed t test.

FIG. 4H is a bar chart showing perirenal fat of 7-9 week old male Ncb5or+/+, +/−, and −/− mice. N=7-9 mice in each group. Error bars designate mean+/−SEM. *=p<0.05, **=p<0.01 and ***=p<0.001, unpaired two-tailed t test.

FIG. 4I is a line graph showing serum triglycerides level of 7-9 week old male Ncb5or+/+, +/, and −/− mice. N=7-9 mice in each group. Error bars designate mean+/−SEM. *=p<0.05, **=p<0.01 and ***=p<0.001, unpaired two-tailed t test.

FIG. 5 is a bar graph showing serum insulin levels of Ncb5or −/− mice in the fed state and the fasting state.

FIGS. 6A-D are photographs of tissue sections showing immunostaining of pancreatic cells with anti-insulin (FIG. 3A, C) or anti-glucogon (FIG. 3B, D) antibodies.

FIG. 7 is a bar chart perirenal fat and serum triglyercides of Ncb5or −/− mice compared to control mice.

FIG. 8 is a scatter graph depicting Levels of serum adiponectin (left) and leptin (right) in Ncb5or −/− mice (open circles) and +/+ mice (solid circles) at two different ages.

FIG. 9A is a bar chart showing insulin release on isolated islets from 4-week-old Ncb5or+/+ and −/− mice. (n: WT=8 and KO=7-8)

FIG. 9B is a bar chart showing total insulin content on isolated islets from 4-week-old Ncb5or+/+ and −/− mice. (n: WT=7 and KO=7).

FIG. 10 is a diagram of functional domains Ncb5or.

FIG. 11 is a schematic representaion showing Ncb5or transfers electrons from NAD(P)H via FAD and heme.

### DETAILED DESCRIPTION

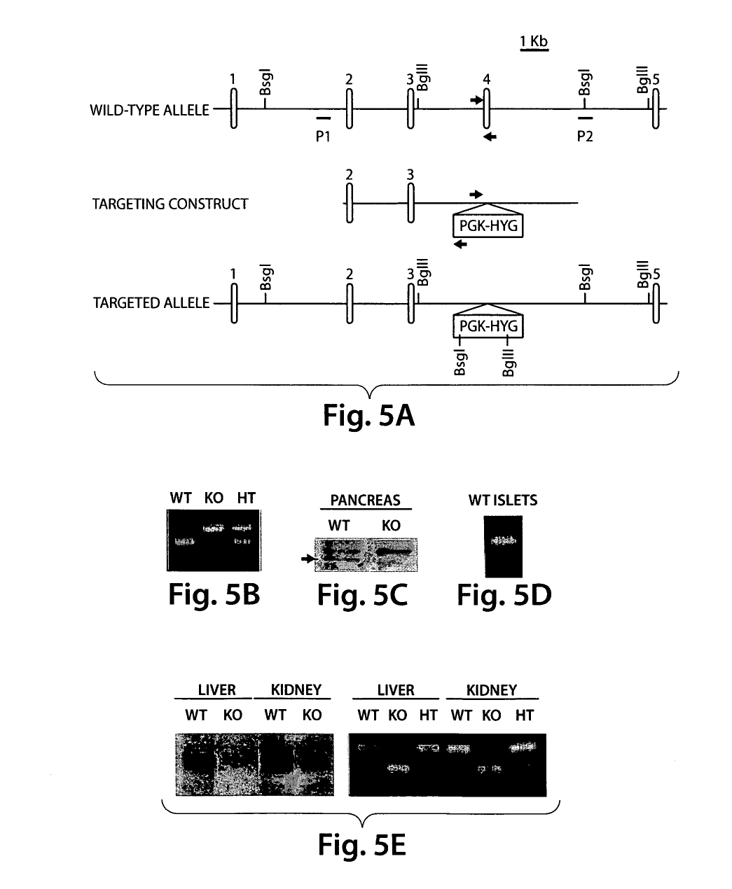

The invention is based upon the unexpected discovery that deletion of the oxidoreductase Ncb5or gene leads to insulin deficiency in mice. Ncb5or is a 56 kDa polypeptide containing a 135-residue N-terminal domain having strong homology to classic microsomal cytochrome b5, a 6-coordinate heme protein. At the C-terminus there is a 262-residue domain with homology to classical microsomal cytochrome b5 reductase, a flavoprotein. These two domains are joined by a 90-residue hinge region essential for enzymatic function. (FIG. 5) Ncb5or is highly conserved in many animals including worms and flies. Specifically, human Ncb5or polypeptide shares 80% sequence identity with murine Ncb5or and 83% sequence identity with rat Ncb5or. Ncb5or is expressed in a wide variety of organs, tissues and cell lines. In particular, Ncb5or is strongly expressed in whole pancreas as well as in insulinoma cell lines. Native and functional Ncb5or, produced in E. Coli, contains a single heme and a single flavin (FAD) moiety. Activities of Ncb5or include reduction of a number of substrates including cytochrome C, methemoglobin, and ferric iron, and conversion of molecular oxygen to superoxide.

Despite its widespread expression, targeted ablation of Ncb5or in mice results in a very specific phenotype: severe diabetes with pronounced impairment of insulin production in β-cells. Specifically, Ncb5or−/− mice have a phenotype similar to maturity onset diabetes in the young (MODY). Animals up to one month of age have normal blood sugar levels, however by 8 weeks of age the mice develop severe hyperglycemia with marked reduction of plasma insulin. The mice are glucose intolerant and are insulin responsive. The animals have a decrease in white adipose tissue and a reduction of body mass of about 15% compared to the littermate controls. In contrast they have normal amounts of brown adipose. In addition, the animals have an increase in serum trigycerides and serum cholesterol.

Therapeutic methods include the steps of administering to a subject or contacting a cell with a compound that increases flavo-heme oxidase-reductase expression or activity. The compound is, e.g., (i) a flavo-heme oxidase-reductase polypeptide or fragment thereof; (ii) a nucleic acid encoding a flavo-heme oxidase-reductase polypeptide or fragment thereof; (iii) a nucleic acid that increases expression of a nucleic acid that encodes a flavo-heme oxidase-reductase polypeptide (e.g., promoters, enhancers); (iv) an agonist of a flavo-heme oxido-reductase polypeptide; or (v) an inducer of the expression of a flavo-heme oxido-reductase nucleic acid (i.e., a compound that upregulates transcription).

As used herein, the term “nucleic acid” includes DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded. The nucleic acid is operably linked to a regulatory sequence. “Operably linked” means that the nucleotide sequence of interest is linked to the regulatory sequence(s) in a manner that allows expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, e.g., in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence preferentially in certain host cells (e.g., tissue-specific regulatory sequences). For example the regulatory sequence directs expression of the nucleic acid in pancreatic islet cell. Alternatively the regulatory sequence is a mammalian insulin promoter, such as a murine, rodent or human insulin promoter. The nucleic acid is endogenous or heterologous.

A flavo-heme oxido-reductase polypeptide or nucleic acid is preferably Ncb5or. Suitable sources of nucleic acids encoding Ncb5or include a human Ncb5or nucleic acid (and the encoded protein sequences) available as GENBANK™ Accession No. AF169803 (SEQ ID NO;1) and AAF04812 (SEQ ID NO:2), respectively. The sequences are illustrated below in Tables 1 and 2. Other sources include rat Ncb5or nucleic acid and protein sequences are shown in GENBANK™ Accession No. XM229210 and XP229210, respectively, and are incorporated herein by reference in their entirety. Additional sources include murine, rat or human b5 or b5R nucleic acid and protein sequences as shown in GENBANK™ Accession No. P56395, P00173, or P20070 incorporated herein by reference in their entirety.

Alternatively, the compound is a fragment of a Ncb5or nucleic acid or polypeptide. For example, the compound has one or more functional domains of a Ncb5or, such as the cyt b5 domain, the cyt brR domain or the hinge region. Preferably, the fragment contains one or more heme or flavin (FAD) moieties. Exemplary fragments include amino acids 1-135 of SEQ ID NO: 2 (SEQ ID NO:3), amino acids 136-225 of SEQ ID NO: 2 (SEQ ID NO:4), or amino acid 226-487 of SEQ ID NO:2(SEQ ID NO:5). (See, Tables 3-5)

TABLE 1
Human Ncb5or nucleic acid sequence (cDNA) (SEQ ID NO: 1)

| | |
|---|---|
| 1 | agccttatgg attggattcg actgaccaaa agtggaaagg atctaacggg attaaaaggc |
| 61 | aggttaattg aagtaactga agaagaactt aagaaacaca acaaaaaaga tgattgttgg |
| 121 | atatgcataa gaggtttcgt ttataatgtc agcccttata tggagtatca tcctggtgga |
| 181 | gaagatgaac taatgagagc agcaggatca gatggtactg aactttttga tcaggttcat |
| 241 | cgttgggtca attatgaatc catgctgaaa gaatgcctgg ttggcagaat ggccattaaa |
| 301 | cctgctgttc tgaaagacta tcgtgaggag gaaaagaaag tcttaaatgg catgcttccc |
| 361 | aagagccaag tgacagatac acttgccaaa gaaggtccta gttatccaag ctatgattgg |
| 421 | ttccaaacag actctttagt caccattgcc atatatacta aacagaagga tatcaattta |
| 481 | gactcaatta tagttgatca tcagaatgat tcctttagag cagaaacaat tattaaggat |
| 541 | tgtttatatc ttatacatat tgggctaagc catgaggttc aggaagattt ttctgtgcgg |
| 601 | gttgttgaga gtgtgggaaa aatagagatt gttctacaaa aaaaagagaa tacttcttgg |
| 661 | gactttcttg gccatcccct gaagaatcat aattcactta ttccaaggaa agatacaggt |
| 721 | ttgtactaca gaaagtgcca gttaatttcc aaggaagatg ttactcatga tacgaggctt |
| 781 | ttctgtttga tgctgccacc aagcactcat cttcaagtgc ccattgggca acatgtttac |
| 841 | ctcaagctac ctattacagg tacagaaata gtaaagccat atacacctgt atctggttcc |
| 901 | ttactctcag agttcaagga accagttctt cccaacaata aatacatcta ctttttgata |
| 961 | aaaatctatc ccactggact cttcacacca gagcttgatc gtcttcagat tggagatttt |
| 1021 | gtttctgtaa gcagtcctga gggcaatttt aaaatatcca agttccaaga attagaagat |
| 1081 | ctctttttgt tggcagctgg aacaggcttc acaccaatgg ttaaaatact gaattatgct |
| 1141 | ttgactgata tacccagtct caggaaagtg aagctgatgt tcttcaataa aacagaagat |
| 1201 | gatataattt ggagaagcca attggagaaa ttagcattta aagataaaag actggatgtt |
| 1261 | gaatttgttc tctcagcacc tatttctgaa tggaatggca aacagggaca tatttcacca |
| 1321 | gctcttcttt ctgaattttt gaaaagaaat ttggacaaat ccaaagttct cgtctgcatt |
| 1381 | tgtggaccag tgccatttac agaacaagga gtaaggttgc tgcatgatct caacttttcc |
| 1441 | aaaaatgaga tccatagttt tacagcataa tgaagagctg tcattgtcct ttattcaact |
| 1501 | agtttatcta aatttgtgat tgcttagggt tttttaagag aacatttttg tacataacaa |
| 1561 | aaggttaact agaatccagc cttcagtttc ttaaatgaaa tcaaatgttc cttcagtaca |
| 1621 | ggtaacttct tggctttctt ttgtaccaca acttatttta ctactgatat ttgacc |

TABLE 2
Human Ncb5or polypeptide sequence (SEQ ID NO: 2)

| | |
|---|---|
| 1 | mdwirltksg kdltglkgrl ievteeelkk hnkkddcwic irgfvynvsp ymeyhpgged |
| 61 | elmraagsdg telfdqvhrw vnyesmlkec lvgrmaikpa vlkdyreeek kvlngmlpks |
| 121 | qvtdtlakeg psypsydwfq tdslvtiaiy tkqkdinlds iivdhqndsf raetiikdcl |
| 181 | ylihiglshe vqedfsvrvv esvgkieivl qkkentswdf lghplknhns liprkdtgly |
| 241 | yrkcqliske dvthdtrlfc lmlppsthlq vpigqhvylk lpitgteivk pytpvsgsll |
| 301 | sefkepvlpn nkyiyfliki yptglftpel drlqigdfvs vsspegnfki skfqeledlf |
| 361 | llaagtgftp mvkilnyalt dipslrkvkl mffnkteddi iwrsqlekla fkdkrldvef |
| 421 | visapisewn gkqghispal lseflkrnld kskvlvcicg pvpfteqgvr llhdlnfskn |
| 481 | eihsfta |

TABLE 3
Human Ncb5or Cyt b5 Domain polypeptide sequence (SEQ ID NO: 3)

mdwirltksg kdltglkgrl ievteeelkk hnkkddcwic irgfvynvsp ymeyhpgged
elmraagsdg telfdqvhrw vnyesmlkec lvgrmaikpa vlkdyreeek kvlngmlpks
qvtdtlakeg psyps

TABLE 4

Human Ncb5or HingeRegion polypeptide sequence
(SEQ ID NO: 4)

ydwfq tdslvtiaiy tkqkdinlds iivdhqndsf raetiikdcl
ylihiglshe vqedfsvrvv esvgkieivl qkkentswdf lghpl

TABLE 5
Human Ncb5or Cyt b5r polypeptide sequence (SEQ ID NO: 5)

knhns liprkdtgly yrkcqliske dvthdtrlfc lmlppsthlq vpigqhvylk
lpitgteivk pytpvsgsll sefkepvlpn nkyiyfliki yptglftpel drlqigdfvs
vsspegnfki skfqeledlf llaagtgftp mvkilnyalt dipslrkvkl mffnkteddi
iwrsqlekla fkdkrldvef visapisewn gkqghispal lseflkrnld kskvlvcicg
pvpfteqgvr llhdlnfskn eihsfta

Human Ncb5or polypeptides shares homology to other members of the flavo-heme oxidase-reductase protein family. The homology between human, murine and rat Ncb5or polypeptides is shown graphically in the ClustalW analysis. (Table 6) The analysis was performed using the program BioEdit. In the ClustalW alignment, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function. The ClustalW alignment was used to generate a Ncb5or consensus sequences as shown in SEQ ID NO:8. Consensus sequences for the Cyt b5 domain (SEQ ID NO:9), hinge region (SEQ ID NO:10) and Cytb5r domain (SEQ ID NO:11) are shown in Tables 6, 7 and 8. Accordingly, the compound includes SEQ ID NO: 8, 9, 10 or 11.

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

[image]

TABLE 7
Human Ncb5or Cyt b5 Domain Consensus sequence (SEQ ID NO: 9)

MDWxRLTKSGKDxTGLKGxLIEVTEEELKKHNKKxDCWICIRGFVYNVSPYMEYHPGGEDELMRAAGxDGTxLFxxVHRW
VNYESMLKECLVGRMAxKPAVxKDxxExxKxVLNGMLPKSQxxxxxxxxVTDTLx

TABLE 8
Human Ncb5or Hinge Region Consensus sequence (SEQ ID NO: 10)

xEGxSxPSYDWFQTxSxVTIxxYTKQKxIxLDSxIVDxQxDSxRAExxIKDxxYLxHxGLSHEVQExFSVRVxExVGKIE
IVLxKKExxSWxxLGxxLxxHxSxI

TABLE 9
Human Ncb5or Cyt b5r Domain polypeptide sequence (SEQ ID NO: 11)

PxKDTGLYYRxCQLISKEDVTHDTRLxCLMLPPSTHLQVPxGQHVYLKLxxTGxEIVKPYTPVSxSLLSxFKEPVLxxNK
YIYFLIKIYPxGLFTPELDRLQIGDFxSVSxPEGNFKxSKxQExEDLFLLAAGTGFTPMVxxLNxALxxxxSLRKVKLMF
FNKTEDDIIWRxQLEKLAxxxKRxxVExVLSAPxxEWNGKQGHxSxALLSEFLxRxxxxSxxxxCICGPxPFTxxGxRLL
HDLNFSxxEIHxFTA

The compound is administered to the subject either directly (i.e., the subject is directly exposed to the nucleic acid or nucleic acid-containing vector) or indirectly (i.e., cells are first transformed with the nucleic acid in vitro, then transplanted into the subject). For example, mammalian cells are isolated from a subject and the flavo-heme oxido-reductase nucleic acid introduced into the isolated cells in vitro. The cells are reintroduced into a suitable mammalian subject. Preferably, the cell is introduced into an autologous subject. The routes of administration of the compound can include e.g., parenteral., intravenous, intradermal, subcutaneous, oral (e.g., inhalation), transdermal (topical), transmucosal, and rectal administration. For example, compound is administered intravenously. Alternatively, transformed cells are surgically transplanted into pancreatic tissue.

The cell can be any cell that is capable of producing insulin For example, the cell is a pancreatic islet cell (i.e., alpha or beta). Alternatively, the cell is a muscle, spleen, kidney, blood, skin, pancreas, or liver cell.

The subject is preferably a mammal. The mammal can be, e.g., a human, non-human primate, mouse, rat, dog, cat, horse, or cow.

The invention provides methods of increasing insulin production by contacting a cell with compound that increases a flavo-heme oxido-reductase polypeptide expression or activity. The cell is any cell that expresses insulin, e.g., the cell is a pancreatic cell such as a pancreatic islet cell.

The invention also provides methods of increasing serum insulin levels or decreasing blood glucose levels. Serum glucose levels are decreased or insulin level are increased in a subject in need thereof. A subject is identified by measuring either blood glucose or insulin levels by methods know in the art. For example by measuring fasting blood glucose levels. A subject is in need of increased serum insulin or decreased blood glucose levels if the subjects insulin or glucose levels are not in normal ranges. Normal adult glucose levels are 60-120 mg/dl. Normal insulin levels are 7 mU/mL±3 mU. For example if the subjects serum glucose levels are greater than 120 mg/dl, the subject requires a decrease in serum glucose level. Preferably, after administration the subjects serum glucose is altered to between 60-120 mg/dl. A subject is in need of increased insulin levels, if serum insulin levels are less than 4 mU/mL. Preferably, after administration serum insulin levels are altered such that serum insulin levels are within a normal range, e.g., 7 mU/mL±3 mU.

A method of treating, preventing or alleviating a symptom of diabetes is carried out by administering to a subject in which such treatment or prevention is desired a composition containing a compound that increases flavo-heme oxidase-reductase expression or activity in an amount sufficient to treat or prevent the disease in the subject. Efficaciousness of treatment is determined in association with any known method for diagnosing or treating diabetes. Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia insulin deficiency or resistance. Alleviation of one or more symptoms indicates that the compound confers a clinical benefit.

To preferentially reduce white fat in a subject in which such treatment or prevention is desired, a compound that decreases flavo-heme oxidase-reductase is administered. For example, the compound is an antisense Ncb5or nucleic acid, a Ncb5or-specific short-interfering RNA, or a Ncb5or-specific ribozyme. Alternatively, the compound an inhibitor of oxidoreductase activity such as an iodonium compound, e.g., diphenyl iodonium. For example the subject is overweight, obese or at risk of becoming overweight or obese. Methods of determining whether or not an individual is overweight or obese are known in the art. For example, Body mass index (BMI) is measured (kg/m² (or lb/in²×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e. fat mass impedes current), estimates % fat) is measured. The parameters for normal, overweight, or obese individuals is as follows: Underweight: BMI<18.5; Normal: BMI 18.5 to 24.9; Overweight: BMI=25 to 29.9. Overweight individuals are characterized as having a waist circumference of >94 cm for men or >80 cm for women and waist to hip ratios of ≧0.95 in men and ≧0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of ≧35.

The invention further provides methods of identifying agents that increase insulin production or decreases fat accumulation by contacting a cell containing a Ncb5or polypeptide or nucleic acid with a test agent. The cell is a pancreatic cell or cell line. The level of oxidase activity in the cell is determined. Oxidase activity is measured by methods know in the art. (Johnson et al. (1998), J. Bio. Chem 273:35147-35152). An increase in activity in the presence of the test agent compared to the level in the absence of the test agent indicates the agent increase insulin production or decrease fat accumulation.

Screening Methods

Inducers of Ncb5or expression are identified by incubating a promoter region operably linked to a reporter sequence with a candidate compound. For example, a Ncbor5 promoter sequence is operably linked to a reporter gene. Reporter gene sequences are known in the art. An increase in transcription of the reporter gene (or an increase in the amount of the reporter gene product) in the presence of the candidate compound compared to the level in the absence of the compound indicates that the compound increases Ncb5or expression. A decrease in the level of expression of the reporter gene or gene product in the presence of the candidate compound compared to the level in the absence of the compound indicates that the compound inhibits Ncb5or expression.

A compound that increases Ncbor5 activity is identified by contacting a cell containing an Ncb5or polypeptide with a candidate compound and measuring Ncb5or activity. Ncb5or activity is measured by detecting production of reactive oxygen species (ROS) according to methods known in the art, e.g., Zhu et al., 1999, PNAS 96:14742-14747; Johnson et al., 1998, J. Biol. Chem. 273:35147-35152. An increase in production of ROS in the presence of the compound compared to the amount detected in the absence of the compound indicates that the compound increases Ncb5or activity. An decrease in production of ROS in the presence of the compound compared to the amount detected in the absence of the compound indicates that the compound inhibits Ncb5or activity.

A fatty acid substrate of Ncb5or is identified by analyzing products of Ncb5or enzyme activity. For example, mixture of fatty acids (e.g., a commercially available mixture of 20 fatty acids) is analyzed by high performance liquid chromatography (HPLC) to generate a baseline profile. Each peak in the profile represents a particular fatty acid in the mixture. Recombinant Ncb5or enzyme is incubated in the presence of a cell extract (which is deficient in Ncb5or) and NAD(P)H as an electron donor. The extract is a tissue extract, e.g., liver tissue extract obtained from a Ncb5or −/− animal. Alternatively, the extract is a cell extract of a cell line (e.g., an insulinoma cell line) in which Ncb5or expression is inhibited. For example, Ncb5or expression was successfully downregulated by an antisense construct (e.g., a small (25 nucleotide) double stranded RNA construct, the sequence of which corresponds to the exon 1 junction sequqnece in the Ncb5or genomic sequence). The mixture of recombinant Ncb5or enzyme, extract, fatty acids, and electron donor is incubated and aliquots tested over time. The products are monitored using HPLC. A decrease in a fatty acid peak in an HPLC profile compared to the baseline profile indicates that the fatty acid is a substrate of Ncb5or or precursor of an Ncb5or substrate.

Diagnostic Methods

The presence of mutation in the coding or regulatory region of a Ncb5or gene indicates a predisposition to develop diabetes or a diagnosis of diabetes. A tissue sample, e.g., blood, is obtained from an individual and nucleic acids extracted from the cells. The DNA is analyzed using known methods and compared to a reference sequence, e.g., the sequence of SEQ ID NO:1 (Ncb5or cDNA) or the sequence of GENBANK™ Accession No. AL034347 (Ncb5or genomic sequence). A difference in the patient-derived sequence (e.g., an insertion, deletion, or substitution) compared to the reference sequence indicates that the patient from which the DNA was obtained is suffering from or at risk of developing a diabetic condition. The mutation is in in the Cyt 5b or Cyt 5br domain. Alternatively, the mutation is in the hinge region.

Therapeutic Administration

Mammals such humans which are have been diagnosed with diabetes, high blood glucose, low serum insulin, or at risk of becoming so, are treated with compounds which increase Ncb5or expression or activity. Alternatively, mammals which are overweight, obese, or at risk of becoming so are treated with compounds which decrease Ncb5or expression or activity.

Ncb5or is therapeutically overexpressed (e.g., by administering an inducing agent) to increase expression from the endogenous gene or by administering DNA (alone or in a plasmid) encoding an Ncb5or gene product under the control of a strong inducible or constitutive promoter. Preferably, the promoter preferentially directs expression of Ncb5or in a pancreatic cell, e.g., transcription is at least 10%, 20%, 50%, 100% more in pancreatic cells compared to the level of transcription in non-pancreatic cells.

For local administration of DNA, standard gene therapy vectors used. Such vectors include viral vectors, including those derived from replication-defective hepatitis viruses (e.g., HBV and HCV), retroviruses (see, e.g., WO 89/07136; Rosenberg et al., 1990, N. Eng. J. Med. 323(9):570-578), adenovirus (see, e.g., Morsey et al., 1993, J. Cell. Biochem., Supp. 17E,), adeno-associated virus (Kotin et al., 1990, Proc. Natl. Acad. Sci. USA 87:2211-2215,), replication defective herpes simplex viruses (HSV; Lu et al., 1992, Abstract, page 66, Abstracts of the Meeting on Gene Therapy, September. 22-26, Cold Spring Harbor Laboratory, Cold Spring Harbor, N.Y.), and any modified versions of these vectors. The invention may utilize any other delivery system which accomplishes in vivo transfer of nucleic acids into eucaryotic cells. For example, the nucleic acids may be packaged into liposomes, e.g., cationic liposomes (Lipofectin), receptor-mediated delivery systems, non-viral nucleic acid-based vectors, erytbrocyte ghosts, or microspheres (e.g., microparticles; see, e.g., U.S. Pat. Nos. 4,789,734; 4,925,673; 3,625,214; Gregoriadis, 1979, Drug Carriers in Biology and Medicine, pp. 287-341 (Academic Press,). Naked DNA may also be administered.

DNA for gene therapy can be administered to patients parenterally, e.g., intravenously, subcutaneously, intramuscularly, and intraperitoneally. DNA or an inducing agent is administered in a pharmaceutically acceptable carrier, i.e., a biologically compatible vehicle which is suitable for administration to an animal e.g., physiological saline. A therapeutically effective amount is an amount which is capable of producing a medically desirable result, e.g., an increase or decrease of a Ncb5or gene product in a treated animal. Such an amount can be determined by one of ordinary skill in the art. As is well known in the medical arts, dosage for any given patient depends upon many factors, including the patient's size, body surface area, age, the particular compound to be administered, sex, time and route of administration, general health, and other drugs being administered concurrently. Dosages may vary, but a preferred dosage for intravenous administration of DNA is approximately 10⁶ to 10²² copies of the DNA molecule.

Ncb5or gene products are administered to the patient intravenously in a pharmaceutically acceptable carrier such as physiological saline. Standard methods for intracellular delivery of peptides can be used, e.g. packaged in liposomes. Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, and intraperitoneal.

Transgenic Animals

A transgenic non-human mammal which lacks a functional Ncb5or gene was produced. Standard methodology for producing a transgenic embryo requires introducing a targeting construct, which integrates by homologous recombination with the endogenous nucleic acid sequence of the targeted gene, into a embryonic stem cells (ES). The ES cells are then cultured under conditions effective for homologous recombination (i.e., of the recombinant nucleic acid sequence of the targeting construct and the genomic nucleic acid sequence of the host cell chromosome). Genetically engineered stem cells that are identified as containing a knockout genotype which comprises the recombinant allele are introduced into an animal, or ancestor thereof, at an embryonic stage using standard techniques (e.g., by microinjecting the genetically engineered embryonic stem (ES) cell into a blastocyst). The resulting chimeric blastocyst is then placed within the uterus of a pseudo-pregnant foster mother for the development into viable pups. The resulting viable pups include chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome which comprises the disrupted target gene to be transmitted to the progeny of these founder animals thereby facilitating the production of transgenic “knockout animals” the genomes of which contain a gene which has been genetically engineered to comprise a null mutation.

### EXAMPLE 1

Characterization of Ncb5or

Expression of Ncb5or

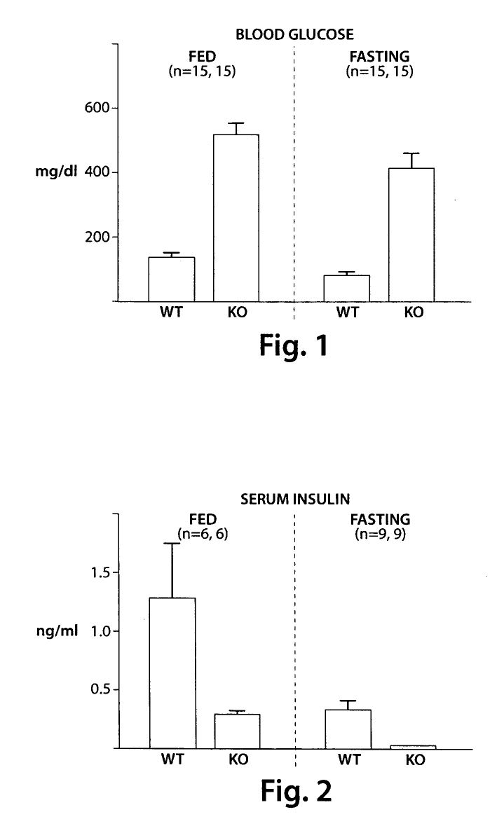

It had previously shown that at the mRNA level, Ncb5or is widely expressed in different organs and tissues of humans (Proc Natl Acad Sci USA 96, 14742-7. (1999)) and mice. As shown in FIG. 1, Ncb5or is also widely expressed in the rat embryo 18 days post conception.

Localization of Endogenous NCB5OR

Confocal microsopyy was performed to determine ssubcellular localization NCB5OR. Human HepG2 hepatoma cells were grown on poly-D-lysine-coated glass cover slips in 24-well dishes over night in DMEM medium. Subconfluent cells were fixed and then blocked with 3% BSA in PBS. Rabbit polyclonal anti-NCB5OR (1:200 dilution) and the chicken polyclonal anti-calreticulin (1:100) were used as primary antibodies. Secondary antibodies were the Alexa-568-conjugated goat anti-rabbit IgG (1:400) and the CY2-conjugated rabbit anti-chicken IgG (1:200). Cover slips were mounted on the slides, and two-photon images of single immunostained cells were captured by a converted microscope (Nikon TE300). Two different fluorescence channels were recorded simultaneously. Image stacks (512×512×64 pixels) were obtained by optical sectioning with a 60× water objective. The fluorescence was registered by two photomultipliers (Hamamatsu PM) and visualized by the EZ 2000 software (Version 2.4.1, Coord Automatisering). Deconvolution of the images was achieved with the Huygens System software (Version 2.2.1, Scientific Volume Imaging) using the Maximum Likelihood Estimation method and the microscopic point-spread function. NCB5OR co-localizes with calreticulin, indicating that it resides primarily in the endoplasmic reticulum. This result is unlikely to be confounded by cross-reaction of the anti-NCB5OR antibody with homologous proteins. It ahs been shown that this antibody has no detectable cross-reactivity with “classic” cytochrome b5 reductase, the protein that that the highest homology with NCB5OR. Moreover, transfected epitope-tagged NCB5OR is also localized to the endoplasmic reticulum. FITC anti-cytochrome C revealed no localization of NCB5OR to the mitochondria. The co-localization results shown in cannot be confounded by antibody crossreactivity.

### EXAMPLE 2

Production of Ncb5or −/− Transgenic Mice

A 9 Kb Ncb5or targeting vector was constructed by replacing exon 4 of the Ncb5or gene with a hygromycin resistance cassette. Exon 4 encodes the heme-binding domain, which is crucial for both enzyme stability and function. (FIG. 2A) Correctly targeted mouse 129 embryonic stem cells were injected into blastocysts obtained from C57BL/6 and Balb/c mice, and germline transmission was documented by Southern blot hybridization as well as by PCR analysis.

Approximately 25% of the offspring of Ncb5or +/− parents were null (−/−) homozygotes. Thus absence of the Ncb5or gene had no detectable impact on embryonic or fetal viability. Although knockout mice do not express NCB5OR protein (FIG. 2c), Northern blots revealed a minute amount of Ncb5or mRNA which lacked exon 4 (FIG. 2e). If translated, this mRNA would encode a short (123 residue) polypeptide which is unlikely to fold into a stable functional protein.

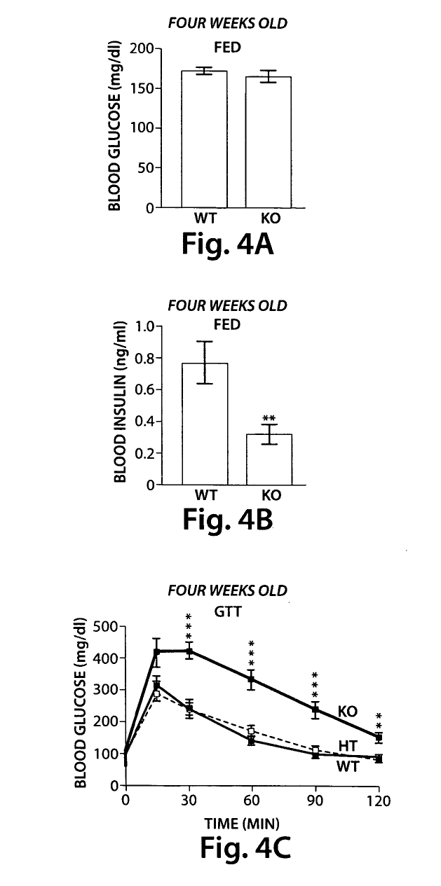

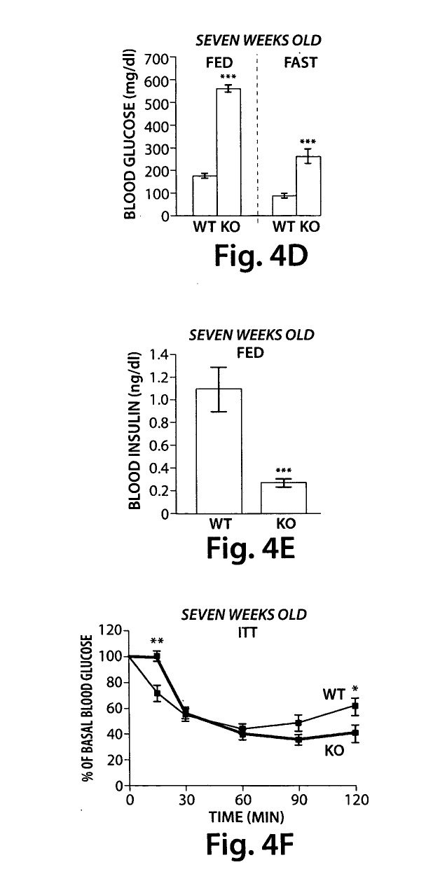

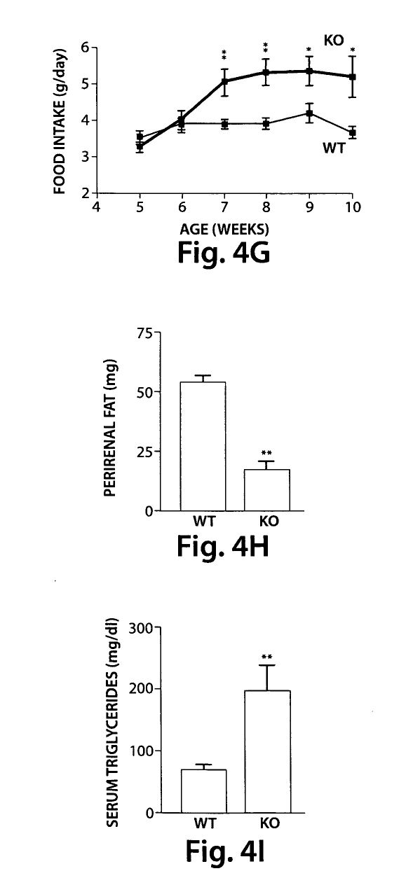

The phenotype of −/− animals was studied in three genetic backgrounds: C57BL/6+129, Balb/c+129, and pure 129, prepared by backcrossing chimeric animals with demonstrated germline transmission of the targeted gene into 129 wild type mice. All of the results presented below pertain to male animals with BALB/cAnN; 129 genetic background. The identical diabetic phenotype has also been seen in male and female C57BL/6;129 Ncb5or −/− and 129 Ncb5or −/− animals. None of the BALB/cAnN;129 Ncb5or −/− mice had any abnormalities on gross or microscopic examination or extensive clinical laboratory evaluation except those noted below. Ncb5or−/+ heterozygotes have normal blood sugar levels and glucose tolerance (FIG. 4c).

### EXAMPLE 3

Characerization of Ncb5or −/− Transgenic Mice

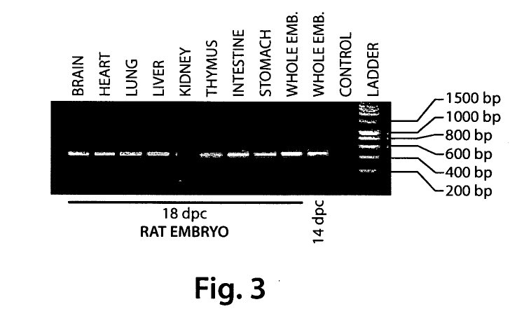

Ncb5or −/− mice have normal blood glucose levels at age 4 weeks but by age 8 weeks develop striking hyperglycemia in both the fed state (morning samples) and following an overnight fast (FIG. 3). These animals have markedly impaired glucose tolerance, as measured by an intraperitoneal glucose tolerance test. At 4 weeks of age, in the fed state, Ncb5or −/− mice had normal blood glucose levels (FIG. 4a) but low insulin levels (FIG. 4b) and impaired glucose tolerance (FIG. 4c) suggesting decreased insulin reserve, i.e. prediabetes. By 7 weeks of age, the blood glucose levels in fed and fasted Ncb5or −/− mice were 3-fold higher than those in Ncb5or +/+ mice (FIG. 4d).

As shown in FIG. 5, in the fed state plasma insulin levels of 8-15 week old animals were decreased to 25% of the levels in normal animals. In fasting −/− animals, insulin was barely detectable in the plasma.

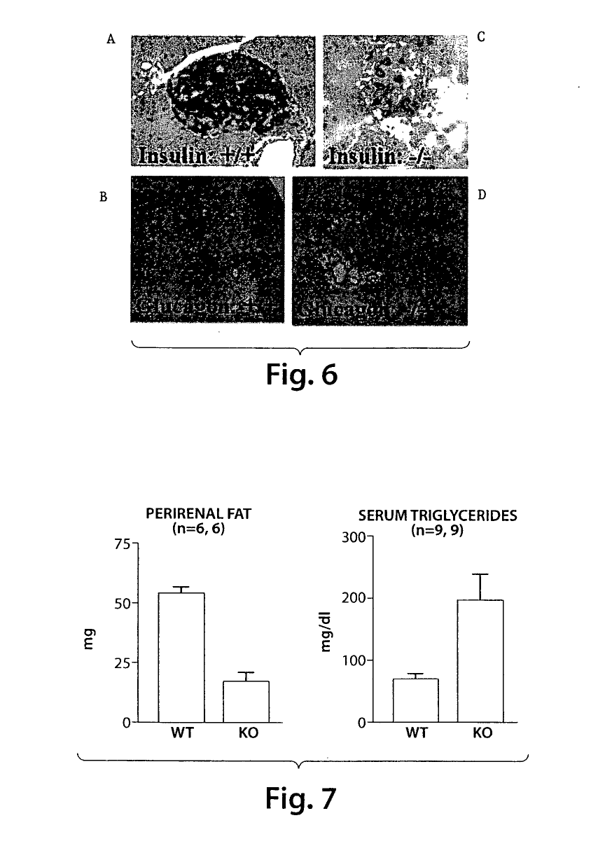

Hematoxylin and eosin-stained sections of the −/− pancreas showed a modest but consistent decrease in the size of the islets and, a readily observed decrease in the cytoplasmic to nuclear ratio of cells within the islets. No inflammatory infiltrates could be seen within the islets. As shown in FIG. 6, immuno-staining with an anti-insulin antibody revealed a striking decrease in the number of positively staining cells. The few cells that were positive showed much less intense staining than β-cells from wild type controls. Staining with an anti-glucagon antibody revealed the expected number and localization of positive cells in the periphery of islets from wild-type mice, whereas in −/− animals a roughly equal number of positive cells were distributed throughout the islets. Similar results were obtained with anti-somatostatin antibody and anti-pancreatic polypeptide antibody. Thus, the Ncb5or −/− animals have a marked deficiency of β cells, whereas the remaining cells in the islets were found to be normal.

In comparison to +/+ littermate controls, −/− animals after 7 weeks of age have a 13±2% decrease in body weight despite a 30±4% increase in food intake. The lower body mass in the −/− animals appears to be due primarily to a reduction in the mass of white adipose tissue (FIG. 7). In contrast, the mass of brown adipose tissue appeared to be normal. No abnormalities were seen in histological sections of either white or brown fat.

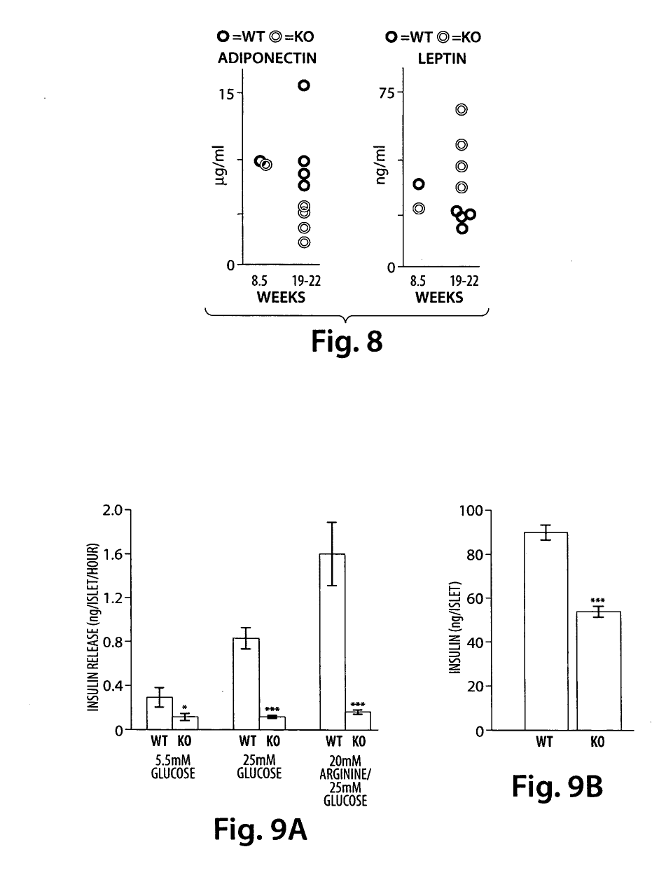

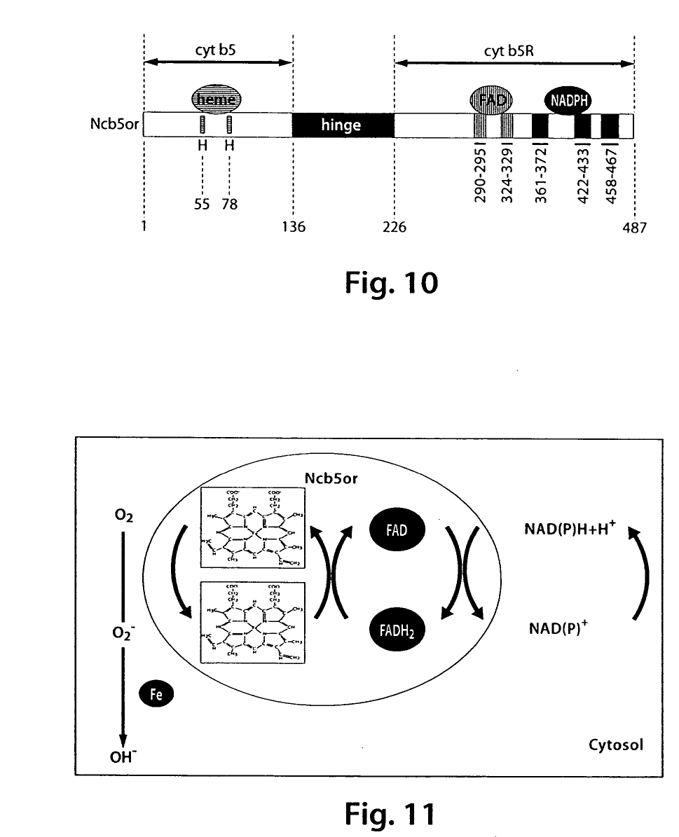

Moreover the livers from −/− animals were functionally and histologically normal with no fatty infiltration. The −/− animals had elevated serum triglycerides (FIG. 7) and cholesterol but normal levels and distribution of plasma free fatty acids. Older (18-22 week old) animals had a ˜50% reduction in serum adiponectin and ˜2-fold elevation in leptin. (FIG. 8) The change in adiponectin levels is not unexpected given the reduced fat mass of Ncb5or −/− animals. The same cannot be said about leptin, however, which increased despite reduced adiposity. This paradox is heightened by the fact that insulin is believed to be a positive regulator of leptin gene expression, with streptozocin treated animals demonstrating markedly reduced leptin mRNA expression in white fat depots. Furthermore, the increased food intake in the setting of hyperleptinemia implies leptin resistance, an uncommon finding in lean animals. These results suggest that hypoinsulinemia alone cannot account for the full complement of metabolic abnormalities in Ncb5or −/− mice.

TUNEL staining on islets of 2- and 4-week old animals showed no significant difference between Ncb5or −/− and +/+ mice. There was also no evidence on either light or electron microscopy of enhanced apoptosis in Ncb5or −/− islets. Moreover, lack of any difference in the expression of Ki-67 protein suggested that inactivation of Ncb5or had no significant effect on cell proliferation in islets. Ncb5or −/− islets were markedly depleted of β-cells , producing an increased volume density of the α, δ, and PP-cell classes. The residual β-cells present in Ncb5or −/− islets were markedly degranulated, with the insulin-containing β granules mostly at the cell periphery, indicative of hypersecretory activity. An unusual feature of these surviving β-cells was an increase in both the number and size of mitochondrial profiles. Many of the mitochondria in β-cells of Ncb5or −/− mice contained electron-dense inclusions. These bodies are likely due to aggregates of a metal, such as iron or calcium.

Isolated 4-week-old Ncb5or −/− islets had a markedly blunted response to 5.5 mM and 25 mM glucose as well as to 20 mM L-arginine/25 mM glucose. Quantitative analysis of the total insulin content showed a 40% decrease in Ncb5or −/− islets compared to sized-matched +/+ islets (FIG. 9b). However, the suppression of insulin secretion in Ncb5or −/− islets greatly exceeded the reduction in insulin content (FIGS. 9a and 7b). This implies that, in these young Ncb5or −/− mice, impaired insulin secretion, rather than decreased biosynthesis, is the earliest β-cell defect.

### EXAMPLE 4

Identifying Substrate and Product of Ncb5or

Spectrophotometric screening assay of NADH-consumption and analysis with HPLC-EC and/or GC-MS are used to identify physiological substrate(s) of oxidoreductase Ncb5or. The initial screening are performed on compounds, which are involved in diabetics and adipogenesis, including major classes of poly-unsaturated fatty acids some of which serve as a ligand for key transcription factors, e.g., HNF4, HNF1, etc. The assays are carried out as follows:

Spectrophotometric Assay of Ncb5or-dependent NADH Consumption

Ncb5or dependent NADH consumption is determined spectrophotometrically as follows:

Step 1: The following extracts are prepared: cytosolic lysate or membranes from liver or insulinoma cells which contain either (1) no (or less than wild type) endogenous b5/b5R (−Ncb5or) or (b) abundant Ncb5or (+Ncb5or) with and without exogenous Ncb5or expressed in E. coli. The extracts provide all accessory factors for Ncb5or enzymatic activity.

Step 2: Measure the NADH-consumption (decrease of OD340) under the following conditions:

a. lysate or membranes (−Ncb5or) or
b. lysate or membranes (−Ncb5or)+potential substrate i (free fatty acid (FFA), etc).
   Calculate the difference between the two slopes, 2b-2a
   Step 3: Measure the NADH-consumption (decrease of OD340) under the following conditions:
a. lysate or membranes (+Ncb5or) or
b. lysate or membranes (+Ncb5or)+potential substrate i (FFA, etc).
   Calculate the Difference the Two Slopes, 3b-3a
   Step 4: The ratio between slope (3b-3a) and slope (2b-2a) is calculated to obtain Ncb5or-dependent NADH-consumption on substrate i.

The compound(s), which showed the highest value in step 4 (with either lysate or membranes) are analyzed using High performance liquid chromatography with electrochemical detection (HPLC-EC).

High Performance Liquid Chromatography with Electrochemical Detection (HPLC-EC)

The conversion of free fatty acid is monitored by HPLC-EC using known methods, e.g., the method described by Kotani, et al (Analytical Biochemistry, 284:65-69, 2000). The substrate and its product(s) catalyzed by b5/b5R are separated on HPLC and monitored under ultra violet and electrochemical detection. A series of standard FFA are obtained commercially and their elution profiles used to identify the product by comparing an HPLC profile of the standard (baseline) FFA to the HPLC profile of FFA after a Ncb5or-catalyzed reaction. Product(s) recovered from HPLC are subject to further characterization with gas chromatography—mass spectrometry (GC-MS).

GC-MS

The size and subclass of Ncb5or-catalyzed product(s) are accurately determined by GC-MS using standard methodology, e.g., as described by Waddington, et al (Analytical Biochemistry, 292:234-44, 2001).™

### EXAMPLE 5

Identification of NCB5OR Mutations

Mutations in the human Ncb5or gene were searched by use of an automated high throughput analysis of PCR products by HPLC. The following three patient groups were screened:
Type 2 diabetic patients (˜60 patients) with a normal disease history and normal phenotypic characteristics.
Probands from MODYX families (˜60 patients), i.e. those in which the mutation is unknown (approximately 60 patients). In these families mutations in HNF1α and GCK have been excluded.
Type 2 diabetic patients with disease age-of-onset below 40 years (˜50 patients)

Two mutations were identified: His223Arg and IVS5+7-8delCT. These sites were then genotyped in 717 type 2 patients and 529 glucose-tolerant control subjects. The respective allele frequencies were ˜0.6% and ˜1.1%. There was no significant association of either of these minor polymorphisms with diabetes.

Mutation in the NCB5or gene in mice were identified by using the NOD mouse model. The NOD mouse model has been very useful for investigating the pathogenesis of type 1 diabetes. Am J Pathol 128, 380-3. (1987). A presumptive diabetes susceptibility gene in NOD mice has been identified at the telomeric end of chromosome 9, the same site as Ncb5or. In order to ascertain whether Ncb5or could be implicated, all of the Ncb5or exons from NOD mice and the control NON strain from which NOD was derived were sequenced. A single difference in the coding region—a missense mutation with Gly in NOD replacing 179Asp in NON (also in NCBI database) was identified. This replacement is within the functionally important hinge region of Ncb5or, and therefore could have an impact on diabetic phenotype. In order to pursue this possibility a NOD stock (N13) congenic for an 18 cM segment of NON in distal chromosome 9 was tested. This limited crossover of NON into NOD resulted in abolition of diabetes susceptibility on chromosome 9. However the Ncb5or gene of this NOD.NON congenic had the NOD sequence (179Gly). Therefore Ncb5or can be ruled out as contributing to the NOD diabetes susceptibility on chromsome 9. The group of Levi-Strauss at L'Institut National de la Sante et de la Recherche Medicale in Paris has identified another locus on mouse chromosome 9 (D9Mit135 marker, 48 cM), associated with NOD susceptibility to high dose streptozotocin-induced diabetes. Our exon analysis has identified the same Asp179Gly missense mutation, indicating that the NCB5OR sequence in the diabetic backcrossed offspring originates from the NOD mouse.

Other embodiments are within the following claims.

1. A method of increasing insulin production, comprising contacting a pancreatic islet cell with a flavo-heme oxido-reductase polypeptide or an agonist thereof.

2. The method of claim 1, wherein said polypepeptide comprises SEQ ID NO: 4.

3. The method of claim 2, wherein said polypeptide further comprises SEQ ID NO: 3.

4. The method of claim 2, wherein said polypeptide further comprises SEQ ID NO: 5.

5. The method of claim 1, wherein said polypeptide or agonist binds to a fatty acid.

6. A method of increasing insulin production, comprising contacting a pancreatic islet cell with a nucleic acid encoding a flavo-heme oxido-reductase polypeptide.

7. The method of claim 6, wherein said nucleic acid is operatively linked to a promoter, wherein said promoter directs expression of said nucleic acid preferentially in pancreatic islet cells compared to non-pancreatic cells.

8. The method of claim 7, wherein said promoter comprises a human insulin promoter.

9. A method of alleviating a symptom of diabetes in a subject, comprising administering to said subject a compound which increases the expression or activity of Ncb5or.

10. The method of claim 9, wherein said compound is a nucleic acid molecule encoding Ncb5or.

11. The method of claim 9, wherein said is an inducer of Ncb5or expression.

12. The method of claim 9, wherein said compound is a Ncb5or polypeptide.

13. A method of increasing insulin production in a cell, the method comprising contacting said cell with a composition which increases the expression or activity of Ncb5or.

14. The method of claim 13, wherein said cell is pancreatic cell.

15. The method of claim 13, wherein said cell is provided in vivo, in vitro or ex vivo.

16. A method of increasing serum insulin levels in a subject, the method comprising administering to said subject a compound which increases the expression or activity of Ncb5or.

17. The method of claim 16, wherein said compound is a nucleic acid molecule encoding Ncb5or.

18. The method of claim 16, wherein said is an inducer of Ncb5or expression.

19. The method of claim 16, wherein said compound is a Ncb5or polypeptide.

20. The method of claim 16, wherein the subject is suffering from or at risk of developing diabetes.

21. A method of decreasing serum glucose levels in a subject, the method comprising administering to said subject a compound which increases the expression or activity of Ncb5or.

22. The method of claim 21, wherein said compound is a nucleic acid molecule encoding Ncb5or.

23. The method of claim 21, wherein said is an inducer of Ncb5or expression.

24. The method of claim 21, wherein said compound is a Ncb5or polypeptide.

25. The method of claim 21, wherein the subject is suffering from or at risk of developing diabetes.

26. A method of diagnosing diabetes or a predisposition thereto, comprising detecting a mutation in a gene encoding Ncb5or, wherein the presence of said mutation indicates a diagnosis or diabetes or a predisposition thereto.

27. A method of diagnosing diabetes or a predisposition thereto, comprising measuring the level of Ncb5or in a patient-derived bodily tissue, wherein a decrease in said level compared to a normal control level, indicates a diagnosis of diabetes or a predisposition thereto.

28. A method of reducing white fat in a subject, comprising administering to said subject a compound which decreases the expression or activity of Ncb5or.

29. The method of claim 28, wherein said compound is selected from the group consisting of a antisense Ncb5or nucleic acid, a Ncb5or-specific short-interfering RNA, and a a Ncb5or-specific ribozyme.

30. The method of claim 28, wherein said compound is an inhibitor of oxidoreductase activity.

31. A method of inhibiting the loss of beta cells in pancreatic islet tissue, comprising contacting said pancreatic islet tissue with a flavo-heme oxido-reductase polypeptide.

32. The method of claim 31, wherein said pancreatic islet tissue comprises at least 10% more beta cells in the presence of said flavo-heme oxido-reductase polypeptide compared to the amount in the absence of said flavo-heme oxido-reductase polypeptide.

33. The method of claim 31, wherein the amount of a reactive oxygen species in said pancreatic islet tissue is reduced in the presence of said flavo-heme oxido-reductase polypeptide compared to the amount in the absence of said flavo-heme oxido-reductase polypeptide.

34. The method of claim 33, herein said reactive oxygen species comprises superoxide (O₂⁻) or ferri-heme.

35. The method of claim 31, further comprising contacting said pancreatic islet tissue with a anti-oxidant.

36. The method of claim 35, wherein said anti-oxidant is a niacin compound.

37. The method of claim 36, wherein said niacin compound is nicotimamide.

38. A method of increasing the viability of primary pancreatic islet cells, comprising contacting said islet cells ex vivo with a flavo-heme oxido-reductase polypeptide or agonist thereof.

39. A method of increasing the viability of transplanted donor pancreatic islet cells in a transplant recipient, comprising administering to said transplant recipient a flavo-heme oxido-reductase polypeptide or agonist thereof.

40. The method of claim 39, wherein said polypeptide or agonist thereof is administered locally to a transplantation site.

41. The method of claim 39, wherein said polypeptide or agonist thereof is administered systemically.

42. The method of claim 39, wherein said polypeptide or agonist thereof is administered prior to transplantation of said donor pancreatic islet cells.

43. The method of 39, wherein said polypeptide or agonist thereof is after transplantation of said donor pancreatic islet cells.

44. The method of 39, wherein said polypeptide or agonist thereof is administered concurrently with transplantation of said donor pancreatic islet cells.

45. A method of inhibiting cell death, comprising contacting a cell with a composition comprising a flavo-heme oxido-reductase polypeptide or an agonist thereof.

46. The method of claim 45, wherein said polypeptide comprises SEQ ID NO: 4.

47. The method of claim 45, wherein said polypeptide further comprises SEQ ID NO: 3.

48. The method of claim 45, wherein said polypeptide further comprises SEQ ID NO: 5.

49. The method of claim 45, wherein said cell is a pancreatic cell.

50. The method of claim 49, wherein said pancreatic cell is a β-cell.

51. The method of claim 45, wherein said cell is provided in vivo, in vitro or ex vivo.

52. The method of claim 45, wherein said cell death is oxidative stress induced cell death.

53. The method of claim 45, wherein said cell death is apoptotic cell death.

54. A pharmaceutical composition comprising a Ncb5or polypeptide.

55. The composition of claim 54, wherein said composition further comprises a fatty acid.

56. A pharmaceutical composition comprising a Ncb5or nucleic acid.

57. A method of identifying an agent that increases insulin production, comprising:
(a) contacting a cell comprising a Ncb5or polypeptide with a test agent; and
(b) determining the level of oxidase activity in said cell, wherein an increase in oxidase activity in the presence of said agent compared to said level in the absence of said agent indicates that said agent increases insulin production.

58. A method of identifying an agent that decreases fat accumulation, comprising:
(a) contacting a cell comprising a Ncb5or polypeptide with a test agent; and
(b) determining the level of oxidase activity in said cell, wherein an increase in oxidase activity in the presence of said agent compared to said level in the absence of said agent indicates that said agent decreases fat accumulation.

59. A transgenic mouse comprising comprising a homozygous disruption in a Ncb5or gene.
